(19) United States
(12) Patent Application Publication
(10) Pub. No.: US 2015/0225358 A1
(43) Pub. Date: Aug. 13, 2015

(54) PROCESS FOR MAKING CERTAIN EPOXIDIZED FATTY ACID ESTER PLASTICIZERS

(71) Applicants: Stephen Howard, Sherman, IL (US); Erik Hagberg, Decatur, IL (US)

(72) Inventors: Stephen Howard, Sherman, IL (US); Erik Hagberg, Decatur, IL (US)

(73) Assignee: ARCHER DANIELS MIDLAND COMPANY, Decatur, IL (US)

(21) Appl. No.: 14/178,692

(22) Filed: Feb. 12, 2014

(51) Int. Cl. C07D 301/00

(52) U.S. Cl. CPC C07D 301/00

(57) ABSTRACT

A process is described for making an epoxidized fatty acid ester material useful as a plasticizer for flexible PVC applications, comprising: transesterifying a low moisture epoxidized natural fat or oil with a first alcohol in a first transesterification step; then, after the resultant product mixture phase separates into an epoxidized fatty acid ester phase and a second phase comprising byproduct glycerol, substantially removing the second phase; combining epoxidized fatty acid esters in the epoxidized fatty acid ester phase with more of the first alcohol and with a second alcohol which includes 5 to 7 members in a ring structure and carrying out a second transesterification step, while continuously removing first alcohol during the second transesterification step in order to drive the reaction with the second alcohol toward the desired epoxidized fatty acid ester material from the second alcohol, but without large molar excesses of the second alcohol being required.

### BACKGROUND OF THE INVENTION

This invention relates to polyvinyl halide plasticizers which have been derived from renewable materials, such as vegetable oil, and more particularly relates to the methods by which such plasticizers are made.

Polyvinyl chloride (PVC), the most common vinyl halide polymer, finds commercial application in a rigid, substantially unplasticized form and in a plasticized PVC form.

Rigid PVC, with which the present invention is not concerned, is used for pipework, ducts and the like in which high chemical resistance is needed but not flexibility or pliability. Plasticized PVC, on the other hand, finds application in films, sheeting, wire and cable coverings, moldings, conveyor belting, toys and hose, in addition to serving as a leather substitute and as a fabric covering for upholstered furniture, automotive seating and other articles.

Broadly speaking, plasticizers are materials which are combined with polymers such as polyvinyl chloride (hereinafter, PVC) to impart flexibility, extensibility and workability or some combination of these attributes to the polymer, as needed for a particular end use. Frequently, a combination of primary and secondary plasticizers is used, with the secondary plasticizers not acting in and of themselves to impart the desired attributes to the PVC but serving to improve the effectiveness of the primary plasticizer(s) and optionally offer other characteristics to a PVC composition in which the materials are incorporated.

Historically, the majority of primary PVC plasticizers have been petroleum-derived phthalates and benzoate compounds, dioctyl phthalate and diisononyl phthalate being notable examples. However, such petroleum-derived plasticizers are frequently expensive to produce and use because of fluctuations in the pricing and availability of petroleum, and are increasingly likely to remain so as petroleum reserves are reduced and new supplies prove more costly and difficult to secure. Further, certain of the petroleum-derived phthalate plasticizers have raised concerns for their potential to disrupt human endocrine activity, and regulatory controls have been established in a number of countries to address these concerns.

Unmodified vegetable oils are largely incompatible with PVC resin, but certain modified derivatives of vegetable oils, such as epoxidized soybean oil (ESO), are compatible with PVC resin and have been actively investigated for use as a lower cost, renewable source-based alternative to the petroleum-based plasticizers, both as primary and secondary plasticizers. The interest in developing useful plasticizers from renewable sources, such as vegetable oils, has developed partly also from the expectation that such materials would be less likely to cause physiological disturbances or other injuries to persons coming into contact with products which require plasticizers in their composition. In recent years, as a result, a number of different renewable source based plasticizers for PVC have been introduced in the literature and in the marketplace.

For example, in copending, commonly-assigned U.S. patent application Ser. No. 13/519,956 from International Application No. PCT/US11/20095, filed Jan. 4, 2011 for “Processes for Making High-Purity Renewable Source-Based Plasticizers and Products Made Therefrom”, now published as US 2012/0277357 (hereafter, “US '357”), we described processes for making certain high purity unsaturated fatty acid esters from alcohols including 5 to 7 members in a ring structure, whether cyclic, heterocyclic or aromatic in character, which esters could then be epoxidized (according to a second aspect) to yield renewable source-based plasticizers for polyvinyl halide polymers, and in particular, for PVC. The plasticizers could be incorporated easily into PVC as primary plasticizers at even plastisol levels, and provided plasticized PVC compositions in turn that exhibited improved and unexpected performance in certain respects.

While epoxidized benzyl esters of unsaturated fatty acids had been described or suggested previously for plasticizing PVC, see for example, U.S. Pat. No. 3,377,304 (epoxidized benzyl soyate) and GB 1,049,100, the known methods of making those benzyl esters and subsequent plasticizers resulted in PVC compositional limitations and performance characteristics which had unfortunately limited the use of such materials only to be secondary plasticizers and thermal stabilizers.

We found that by preparing the indicated unsaturated fatty acid esters (including, of course, benzyl soyate esters), whether by reacting alcohols including 5 to 7 members in a ring structure with unsaturated fatty acid lower alkyl esters having low residual monoglycerides and diglycerides or by reacting the alcohols with an unsaturated fatty acid feed having a correspondingly low monoacylglycerol and diacylglycerol content, these limitations could be overcome.

Unfortunately, while the epoxidized benzyl ester plasticizers we made worked very well and exhibited desirable performance characteristics in PVC compositions incorporating the same, benzyl alcohol as a raw material does have two principal drawbacks, in that it is comparatively expensive and in that excess or residual benzyl alcohol (a number of molar excesses of benzyl alcohol were needed to drive either reaction to completion) is very difficult to remove from the EBS plasticizer product.

More recently, in copending, commonly-assigned International Application No. PCT/US12/60497, filed on Oct. 17, 2012 for “Making Epoxidized Esters from Epoxidized Natural Fats and Oils” and claiming priority from U.S. Ser. No. 61/548,757 filed Oct. 19, 2011 (hereafter, the “WO '497 application”), we discovered that fewer excess molar equivalents of alcohol were needed to drive the reaction to completion in making epoxidized fatty acid esters from already epoxidized natural fats and oils, provided an epoxidized natural fat or oil were selected and used having a sufficiently low moisture content. Thus, when a low moisture ESO was reacted with methanol to make an epoxidized methyl soyate ester plasticizer product, the product mixture phase-separated into an epoxidized fatty acid ester phase and a second phase comprising byproduct glycerol, whereas use of a higher moisture ESO provided no phase separation. By removing the byproduct glycerol phase as it was formed (in the low moisture ESO example), consequently, the thermodynamic equilibrium could be shifted toward the product side without needing a large number of molar excesses of the alcohol reactant.

Because benzyl alcohol is both considerably more costly compared to methanol, and also more difficult to separate out from an epoxidized benzyl ester product as compared to excess methanol from a corresponding epoxidized methyl ester product, we considered that perhaps the drawbacks mentioned above for the EBS plasticizer product might be overcome by starting with a low moisture epoxidized soy oil and reacting benzyl alcohol directly with the ESO in the presence of an effective esterification catalyst and under suitable conditions. Unfortunately, however, the byproduct glycerol did not so readily phase-separate in making the EBS plasticizer product from ESO directly (as compared to making EMS from ESO), and the desired product yields could not be realized without still requiring more benzyl alcohol than could be afforded as a raw material or as a residual impurity in the EBS plasticizer product needing to be removed.

### SUMMARY OF THE INVENTION

The present invention concerns the discovery that the desired unsaturated fatty acid esters, namely, the unsaturated fatty acid esters of alcohols including 5 to 7 members in a ring structure, whether cyclic, heterocyclic or aromatic in character, can be made more efficiently by an alternate process to those described in the prior US '357 application. The alternate method makes use of the process of the WO '497 application, but as a first step only. Surprisingly, the product from the first step can be used directly in the second step for forming the subject unsaturated fatty acid esters without further refinement or purification.

More particularly, according to the alternate process of the present invention generally, a low moisture epoxidized natural fat or oil is transesterified with a first alcohol in the presence of a transesterification catalyst and under conditions which are effective for carrying out the transesterification reaction, whereby the resultant product mixture phase-separates into an epoxidized fatty acid ester phase and a second phase comprising byproduct glycerol; the byproduct glycerol phase is substantially removed; the epoxidized fatty acid ester phase is combined with more of the first alcohol and with a second alcohol which includes 5 to 7 members in a ring structure in the presence of a transesterification catalyst and under conditions which are effective for forming epoxidized fatty acid esters of the second alcohol; and the first alcohol is continuously removed from the process under reduced pressure conditions as it is displaced by the second alcohol. In preferred embodiments, the epoxidized fatty acid ester phase containing epoxidized fatty acid esters of the first alcohol is used directly and without any intervening refining or purification step in the transesterification with the second alcohol. In certain embodiments, borohydride is added in the first step of the process for providing reduced color materials, or to both of the first and second steps.

Parenthetically, those skilled in the art will understand in reference to the above summary that in speaking of “a” low moisture epoxidized natural fat or oil, “a” first alcohol, “a” catalyst and “a” second alcohol, the article “a” should not be taken as implying, for example, that more than one of the 5 to 7 member ring structure second alcohols are excluded, that a plurality of low moisture epoxidized fats or oils may not be used and so forth.

### DETAILED DESCRIPTION OF PREFERRED EMBODIMENTS OF THE INVENTION

The alternate process of the present invention for making the epoxidized unsaturated fatty acid esters of alcohols including 5-7 members in a ring structure, for example, epoxidized benzyl soyate esters, fundamentally begins with a reduced alcohol demand process such as described in the WO '497 application.

The reduced alcohol demand process of the WO '497 application uses a low moisture epoxidized natural fat or oil as a starting material. “Low moisture” in the context of the WO '497 application, as well as the present invention, means that the moisture content of the epoxidized natural fat or oil is sufficiently low that the transesterification products will phase separate with time. The degree of “dryness” necessary for a given epoxidized natural fat or oil can be expected to vary somewhat for different epoxidized natural fats and oils, different alcohols or combinations of alcohols, varying transesterification conditions etc., but as a general guideline the moisture content should ordinarily be about 0.5 percent by weight or less, preferably about 0.25 percent by weight or less and more preferably about 0.1 percent by weight or less, as determined by Karl Fischer titration analysis or by any other conventionally practiced measurement method. These moisture contents, it should be noted, generally correspond to those we expect should be suitable given the use of first alcohols to be combined with the epoxidized natural fat or oil which are similarly “dry”, for example, containing about 2500 ppm by weight or less of water, and preferably about 1000 ppm by weight or less as used in the examples which follow.

The required low moisture content for the reduced alcohol demand process of the WO '497 application may be found in certain epoxidized natural fats or oils without any need for further drying. However, other epoxidized natural fats or oils may be found to have excessive moisture, for example, through prolonged exposure to humid storage environments or through other causes, and will need to undergo a drying step in order to provide the desired phase separation of the transesterification products. In the alternative, an epoxidized natural fat or oil having the requisite low moisture content can be made as needed, rather than or in addition to drying a preexistent epoxidized natural fat or oil supply that has been found to contain too much moisture. As well, a low moisture epoxidized natural fat or oil feedstock can be made merely by blending epoxidized natural fats and oils of varying higher and lower moisture contents, to achieve a blended product that qualifies as a low moisture epoxidized natural fat or oil.

As already mentioned, various methods have been published in the literature for drying epoxidized natural fats and oils. Any of the methods that have been found suitable for drying the fats and oils to an extent whereby these fats and oils would properly be characterized as “low moisture” can be used, but an example would involve exposing the epoxidized natural fat or oil to temperatures in the range of from about 90 degrees Celsius to about 130 degrees Celsius for from about 30 to about 60 minutes, under high vacuum conditions. A drying method of this general character is described in U.S. Pat. No. 2,978,463 to Kuester et al., for example, now incorporated herein by reference.

The first, reduced alcohol transesterification step may otherwise be carried out according to conventionally practiced transesterification methods, or may be conducted according to the reduced color transesterification methods described in commonly-assigned, copending International Application No. PCT/US12/38760, filed May 21, 2012 for “Reduced Color Epoxidized Esters from Epoxidized Natural Fats and Oils” and claiming priority from U.S. Ser. No. 61/501,312 filed Jun. 27, 2011, such prior commonly-assigned application also being incorporated by reference.

Accordingly, the epoxidized natural fat or oil can be derived from animal or plant (including vegetable) sources. Preferably the epoxidized natural fat or oil is a vegetable or seed oil, for example, genetically modified oil, soybean oil, linseed oil, corn oil, sunflower oil, canola oil, rapeseed oil, coconut oil, palm kernel oil, palm oil, cottonseed oil, peanut oil, olive oil, tall oil, safflower oil and derivatives and mixtures thereof. Preferably, the oil is a polyunsaturated oil selected from the group above. Most preferably, the polyunsaturated oil is low in C18:3 or higher fatty acids. Although any polyunsaturated oil that has sufficiently low levels of C18:3 or higher fatty acids is suitable for the present method, preferably, the oil is safflower oil, sunflower oil or corn oil. Preferred oils contain less than about 2 percent of C18:3 or higher polyunsaturated fatty acids. More preferably, the oils contain less than about 1 percent of C18:3 or higher polyunsaturated fatty acids. Also preferred are polyunsaturated oils containing less than about 2 percent linolenic acid. More preferably, the linolenic content is less than about 1 percent.

The first alcohol reactant for the first, reduced alcohol transesterification step may broadly be selected from any of the wide variety of aliphatic or cyclic monohydric, dihydric or polyhydric alcohols that will form an epoxidized fatty add ester with the epoxidized natural fats or oils in the presence of a transesterification catalyst, though aromatic alcohols are less preferred. As demonstrated by Kuester et al., unsubstituted aliphatic alcohols as well as amine substituted aliphatic alcohols having an amine group with no reactive hydrogens on the amine nitrogen may also be considered, triethanolamine being an example of the latter. Monohydric aliphatic alcohols having from 1-20 carbon atoms are preferred, and while primary, secondary and tertiary alcohols may he considered, primary monohydric aliphatic alcohols are more preferred. Methanol is particularly preferred.

The catalyst can be any of a number of known transesterification catalysts. Preferably, the catalyst used in the present process is an alkaline catalyst. More preferably, the catalyst is selected from the group consisting of sodium methoxide, sodium ethoxide, potassium methoxide, potassium ethoxide, potassium tert-butoxide or an N-heterocyclic carbene catalyst such as 1,3-Bis(2,6-diisopropylphenyl)imidazol-2-ylidene (CAS 244187-81-3), from Sigma-Aldrich Co. (though other N-carbene catalysts and preparation methods will be within the capabilities of those skilled in the art without undue experimentation). Most preferably, the catalyst used in the present process is sodium methoxide.

The first, reduced alcohol transesterification step is preferably conducted with a low moisture epoxidized natural fat or oil in the presence of a borohydride, according to the reduced color transesterification methods described in commonly-assigned, copending international Application No. PCT/US12/38760, filed May 21, 2012 for “Reduced Color Epoxidized Esters from Epoxidized Natural Fats and Oils” and claiming priority from U.S. Ser. No. 61/501,312 filed Jun. 27, 2011. In one embodiment (described in greater detail in the prior application), borohydride is included in a transesterification reaction mixture with the low moisture epoxidized natural fat or oil and the first alcohol before a transesterification catalyst is introduced. In another embodiment, the borohydride and the catalyst are concurrently or substantially concurrently incorporated in the reaction mixture with the low moisture epoxidized natural fat or oil and the first alcohol. In yet another embodiment, borohydride is incorporated in the reaction mixture both prior to and concurrently with the introduction of the catalyst.

In any of these modes of incorporating borohydride into the transesterification process, the borohydride material can be selected from the group consisting of sodium borohydride, potassium borohydride and lithium borohydride. Preferably, the borohydride is present in an amount between about 1.0 percent and about 0.0001 percent by weight of the reactants and catalyst. More preferably, the amount of borohydride is between about 0.1 percent to about 0.001 percent. The catalyst in any event preferably comprises a greater part of the reaction mixture as compared to the borohydride.

In terms of the process conditions used, the combined low moisture epoxidized natural fat or oil and first alcohol are heated in the presence of the transesterification catalyst (and borohydride, where the reduced color process is used) to effect a transesterification of the low moisture epoxidized natural fat or oil. Preferably, the combined starting materials are heated to a temperature between about 40° C. and about 70° C. under a slight vacuum in an inert atmosphere, such as N2, Ar or CO2. More preferably, the temperature range is from about 40° C. to about 55° C. The reactants are preferably used neat. and the reaction is carried out in the substantial absence of moisture from other sources than the low moisture epoxidized natural fats and oils, with continuous agitation. It is preferred that the atmosphere is free of O2 and is composed of an inert gas such as those listed above. The combined mixture is heated slowly to the above temperature range. During the process of transesterification, the temperature is maintained in the above range until a certain conversion to product has occurred. In one embodiment, at one or more intermediate stages short of full conversion, additional amounts of the first alcohol and/or of the transesterification catalyst (and additional borohydride, if used) can be added for one or more further stages of reaction leading toward a substantially full to full conversion of the low moisture epoxidized natural fat or oil feedstock. In another embodiment, the first alcohol and catalyst (and optional borohydride) are incorporated in one stage, and the reaction continues with the initially incorporated materials until also substantially completed.

In the prior, reduced alcohol work, neutralization and washing of the resultant product mixture are then prescribed; in the context of the present invention, however, the added expense of these additional steps may surprisingly be avoided; after allowing the product of the first, reduced alcohol transesterification step to phase separate into an epoxidized fatty acid ester phase and a byproduct glycerol phase and subsequently removing the byproduct glycerol phase, the remaining epoxidized fatty acid ester phase may be used directly as a feed for the subsequent step of forming the desired unsaturated fatty acid esters of alcohols including 5 to 7 members in a ring structure.

In that second step, the epoxidized fatty acid ester phase containing epoxidized esters of the first alcohol (especially, epoxidized methyl soyate esters formed by reacting methanol with low moisture epoxidized soybean oil) is combined with an additional amount of the first alcohol (preferably anhydrous) and with a second alcohol which includes 5 to 7 members in a ring structure (also preferably anhydrous) in the presence of a transesterification catalyst and under conditions which are effective for forming epoxidized fatty acid esters of the second alcohol, with the first alcohol being continuously removed from the process under reduced pressure conditions to both recover the first alcohol for reuse in the first step of the overall process and to help drive the second esterification reaction toward the desired epoxidized fatty acid ester product. A large molar excess of comparatively costly benzyl alcohol is consequently not needed, and in those embodiments wherein borohydride is included in the second step as well as the first, the borohydride additionally enables higher temperatures to be used without a significant increase in color, further reducing benzyl alcohol requirements.

More particularly, whereas typically up to about 20 molar equivalents of benzyl alcohol were needed (relative to ESO) in the process of the prior US '357 application to drive the reaction to substantial completion, in the process of the present invention typically only from about 6 to about 2 molar equivalents, preferably from about 4 to about 2.5 molar equivalents and more preferably from about 3.3 to about 3 molar equivalents of benzyl alcohol are used in processes conducted according to the most preferred conditions described herein (again relative to ESO). Because some costly benzyl alcohol inevitably remains even at the lower molar excesses contemplated by the present invention and this is either removed with the methanol or appears undesirably to add (albeit at low levels) across the epoxide ring, and particularly since we have found that some residual EMS can remain without materially adversely affecting plasticizer performance characteristics, in general we consider it preferable to have some residual EMS present rather than use larger molar excesses of the benzyl alcohol in trying to achieve full or substantially full conversion, even where the first alcohol is continuously removed to assist in achieving that full or substantially full conversion.

Exemplary second alcohols including five to seven membered ring structures include, but are not limited to, the following: benzyl alcohol (CAS 100-51-6); 2-chlorobenzenemethanol (CAS 17849-38-6); 3-chlorobenzenemethanol (CAS 873-63-2); 4-chlorobenzenemethanol (CAS 873-76-7); 2-bromobenzenemethanol (CAS 18982-54-2); 3-bromobenzenemethanol (CAS 15852-73-0); 4-bromobenzenemethanol (CAS 873-75-6); 2-methoxybenzenemethanol (CAS 612-16-8); 3-methoxybenzenemethanol (CAS 6971-51-3); 4-methoxybenzenemethanol (CAS 105-13-5); 2-furanmethanol (CAS 98-00-0); 3-furanmethanol (CAS 143632-21-7); 5-methyl-2-furanmethanol (CAS 3857-25-8); tetrahydro-2-furanmethanol (CAS 97-99-4); tetrahydro-3-furanmethanol (CAS 15833-61-1); tetrahydro-5-(methoxymethyl)furfuryl alcohol (CAS 872303-99-6); tetrahydro-2H-pyran-2-ol (CAS 694-54-2); tetrahydro-2H-pyran-3-ol (CAS 19752-84-2); tetrahydro-2H-pyran-4-ol (CAS 208144-9); tetranydro-2H-pyran-2-methanol (CAS 100-72-1); tetrahydro-2H-pyran-3-methanol (CAS 14774-36-8); tetrahydro-2H-pyran-4-methanol (CAS 14774-37-9); 1,4;3,6-dianhydro-2-O-methylhexitol (CAS 1175065-15-2) and 1,4:3,6-dianhydro-2-deoxyhexitol (CAS 1078712-23-8).

In one preferred embodiment, an epoxidized soy methyl ester phase from reacting methanol with a low moisture epoxidized soybean oil in the presence of a transesterification catalyst and borohydride, allowing a byproduct glycerol phase to be formed and then removing the byproduct glycerol phase is combined with additional anhydrous methanol, for example, about 0.3 percent by weight of additional anhydrous methanol based on the amount of epoxidized soybean oil starting material, with borohydride and with anhydrous benzyl alcohol in the presence of an alkaline transesterification catalyst. An effective, non-optimized amount of borohydride added in the first and second steps appears for this preferred embodiment to be about 0.3 percent by weight based on the combined reactants and catalyst, though with further optimization of borohydride addition levels we expect it may be found that the borohydride addition for the first step alone will be sufficient, and borohydride addition in the second step omitted if desired.

The alkaline transesterification catalyst can be a sodium methoxide, potassium tert-butoxide or N-heterocyclic carbene catalyst. An example of a commercially available, suitably stable N-carbene catalyst (under air-free conditions) is 1,3-Bis(2,6-diisopropylphenyl)imidazol-2-ylidene (CAS 244187-81-3), from Sigma-Aldrich Co., though other N-carbene catalysts and preparation methods will be within the capabilities of those skilled in the art without undue experimentation. A sodium methoxide catalyst is most preferred.

The reaction is preferably carried out in the presence of the selected catalyst under reduced pressure, with neat reactants insofar as possible, with agitation and in the absence of moisture, with continuous and preferably complete removal of the methanol as the second ester is formed to help drive the reaction toward the desired fatty acid ester plasticizer product.

The alkaline catalyst is then preferably neutralized with acid, for example, with citric acid or phosphoric acid, and the epoxidized unsaturated fatty acid esters of the second alcohols are preferably then washed with water in one more iterations followed by evaporating or stripping away residual water from the washes, adjusting the conditions as necessary to remove any undesired residual alcohol from the second step.

The epoxidized esters can be contemplated for use as primary or secondary plasticizers in a variety of polymers, including halogenated polymers, acid-functionalized polymers, anhydride-functionalized polymers, and nitrile rubbers. An exemplary halogenated polymer is a PVC polymer, where “PVC” or “polyvinyl chloride” as used herein is understood to cover the range of homo- and copolymers of vinyl chloride with typically up to about 20% of comonomers such as vinyl acetate, propylene, ethylene, diethyl maleate, dimethyl fumarate and other ethylenically unsaturated comonomers. Examples of other halogenated polymers include polyvinyl halide polymers, chlorinated polyolefins and chlorinated rubbers. Suitable acid-functionalized polymers include acrylic acid-functionalized polymers, as well as acrylic and other polymers in need of plasticization to reduce glass transitions or improve toughness.

The present invention is further illustrated by the following, non-limiting example:

### EXAMPLE 1

Low moisture epoxidized soybean oil was added to a reactor, which was then purged of air with argon. To the ESO was added a mixture of sodium methoxide (0.3 percent by weight, based on the amount of ESO) and sodium borohydride (0.05 percent by weight) in methanol, using 3.5 molar equivalents of methanol to the amount of ESO. The mixture was stirred and the reaction carried out under argon for one hour, at which point the stirring was stopped. After 15 minutes, a bottom layer of glycerol formed and was removed. The excess methanol was removed under reduced pressure at which point additional glycerol separated from the mixture and was removed.

To the remainder was added a mixture of sodium methoxide (0.3 percent by weight) and sodium borohydride (0.05 percent by weight) in methanol (2 percent of the total mass). To this was further added 3.5 molar equivalents of benzyl alcohol, based on the ESO originally added to the reactor. The reaction was carried out with stirring for 4 hours under vacuum to remove methanol as it was liberated.

The product was allowed to cool under vacuum to room temperature, was neutralized with a citric acid solution, washed three times with deionized water and dried under vacuum.

What is claimed is:

1. A process for making an epoxidized fatty acid ester material, comprising:
transesterifying a low moisture epoxidized natural fat or oil by combination with a first alcohol in the presence of a transesterification catalyst and under conditions which are effective for carrying out the transesterification reaction;
after the resultant product mixture from the reaction of the first alcohol and low moisture epoxidized natural fat or oil phase separates into an epoxidized fatty acid ester phase and a second phase comprising byproduct glycerol, substantially removing the second phase;
combining epoxidized fatty acid esters in the epoxidized fatty acid ester phase from the first transesterification step with more of the first alcohol and with a second alcohol which includes 5 to 7 members in a ring structure in the presence of a transesterification catalyst and under conditions which are effective for forming epoxidized fatty acid esters of the second alcohol, in a second transesterification step; and
continuously removing first alcohol during the second transesterification step.

2. A process according to claim 1, wherein the epoxidized fatty acid ester phase from the first transesterification step is used directly in the second transesterification step, without any intervening step to refine, purify or isolate epoxidized fatty acids from the epoxidized fatty acid ester phase.

3. A process according to either claim 1 or claim 2, wherein in at least one of the transesterification steps, borohydride is included in an amount sufficient to provide a reduced color epoxidized fatty acid ester material from the second transesterification step as compared to an epoxidized fatty acid ester material produced from the second transesterification step under identical conditions but in the absence of borohydride.

4. A process according to claim 3, wherein the first alcohol comprises a monohydric aliphatic alcohol having from 1-20 carbon atoms.

5. A process according to claim 1, wherein the first alcohol comprises a monohydric aliphatic alcohol having from 1-20 carbon atoms.

6. A process according to claim 5, wherein the first alcohol is methanol.

7. A process according to claim 3, wherein the epoxidized natural fat or of contains less than about 2 percent of C 18:3 or higher polyunsaturated fatty acids.

8. A process according to claim 7, wherein the epoxidized natural fat or oil contains less than about 1 percent of C 18:3 or higher polyunsaturated fatty acids.

9. A process according to claim 3, wherein the epoxidized natural fat or oil is from a polyunsaturated oil containing less than about 2 percent linolenic acid.

10. A process according to claim 9, wherein the epoxidized natural fat or oil is from a polyunsaturated oil containing less than about 1 percent linolenic acid.

11. A process according to claim 1, wherein the epoxidized natural fat or oil contains less than about 2 percent of C 18:3 or higher polyunsaturated fatty acids.

12. A process according to claim 1, wherein the epoxidized natural fat or oil is from a polyunsaturated oil containing less than about 2 percent linolenic acid.

13. A process according to claim 3, wherein the amount of borohydride included is from about 0.0001 percent to about 1.0 percent by combined weight of the reactants and catalyst used in a transesterification step.

14. A process according to claim 13, wherein borohydride is included in the first transesterification step or in the second transesterification step, but not in both steps.

15. A process according to claim 3, wherein the second alcohol is selected from the group consisting of benzyl alcohol, 2-chlorobenzenemethanol, 3-chlorobenzenemethanol, 4-chlorobenzenemethanol, 2-bromobenzenemethanol, 3-bromobenzenemethanol, 4-bromobenzenemethanol, 2-methoxybenzenemethanol, 3-methoxybenezenemethanol, 4-methoxybenzenemethanol, 2-furanmethanol, 3-furanmethanol, 5-methyl-2-furanmethanol, tetrahydro-2-furanmethanol, tetrahydro-3-furanmethanol, tetrahydro-5-(methoxymethyl)furfuryl alcohol, tetrahydro-2H-pyran-2-ol, tetrahydro-2H-pyran-3-ol, tetrahydro-2H-pyran-4-ol, tetrahydro-2H-pyran-2-methanol, tetrahydro-2H-pyran-3-methanol, tetrahydro-2H-pyran-4-methanol, 1,4:3,6-dianhydro-2-O-methylhexitol and 1,4:3,6-dianhydro-2-deoxyhexitol.

16. A process according to claim 15, wherein the second alcohol is benzyl alcohol.

17. A process according to claim 16, wherein no more than about 6 molar equivalents of benzyl alcohol are used relative to the initial low moisture epoxidized natural fat or oil supplied to the first transesterification step.

18. A process according to claim 17, wherein no more than about 4 molar equivalents of benzyl alcohol are used relative to the initial low moisture epoxidized natural fat or oil supplied to the first transesterification step.

19. A process according to claim 18, wherein no more than about 3.3 molar equivalents of benzyl alcohol are used relative to the initial low moisture epoxidized natural fat or oil supplied to the first transesterification step.

20. A process according to claim 1, wherein the second alcohol is selected from the group consisting of benzyl alcohol, 2-chlorobenzenemethanol, 3-chlorobenzenemethanol, 4-chlorobenzenemethanol, 2-bromobenzenemethanol, 3-bromobenzenemethanol, 4-bromobenzenemethanol, 2-methoxybenzenemethanol, 3-methoxybenzenemethanol, 4-methoxybenzenemethanol, 2-furanmethanol, 3-furanmethanol, 5-methyl-2-furanmethanol, tetrahydro-2-furanmethanol, tetrahydro-3-furanmethanol, tetrahydro-5-(methoxymethyl)furfuryl alcohol, tetrahydro-2H-pyran-2-ol, tetrahydro-2H-pyran-3-ol, tetrahydro-2H-pyran-4-ol, tetrahydro-2H-pyran-2-methanol, tetrahydro-2H-pyran-3-methanol, tetrahydro-2H-pyran-4-methanol, 1,4:3,6-dianhydro-2-O-methylhexitol and 1,4:3,6-dianhydro-2-deoxyhexitol.

21. A process according to claim 20, wherein the second alcohol is benzyl alcohol.

22. A process according to claim 21, wherein no more than about 6 molar equivalents of benzyl alcohol are used relative to the initial low moisture epoxidized natural fat or oil supplied to the first transesterification step.

23. A process according to claim 22, wherein no more than about 4 molar equivalents of benzyl alcohol are used relative to the initial low moisture epoxidized natural fat or oil supplied to the first transesterification step.

24. A process according to claim 23, wherein no more than about 3.3 molar equivalents of benzyl alcohol are used relative to the initial low moisture epoxidized natural fat or oil supplied to the first transesterification step.

25. A process according to claim 21, wherein the first alcohol is methanol.

26. A process according to claim 3, wherein the borohydride is included in a transesterification reaction mixture prior to or substantially concurrently with the introduction into the mixture of a transesterification catalyst, or both prior to and substantially concurrently with the introduction of a catalyst.

27. A process according to claim 3, wherein at least the first transesterification step is carried out in the substantial absence of moisture from other sources than the low moisture epoxidized natural fat or oil.

28. A process according to claim 27, wherein the first transesterification step is carried out until substantially completed.

29. A process according to claim 27, wherein both transesterification steps are carried out in the substantial absence of moisture from other sources than the low moisture epoxidized natural fat or oil.

30. A process according to claim 27, wherein at least the first transesterification step is additionally carried out under reduced pressure and with an inert gas atmosphere.

31. A process according to claim 27, wherein both transesterification steps are carried out under reduced pressure and with an inert gas atmosphere.

32. A process according to claim 1, wherein at least the first transesterification step is carried out in the substantial absence of moisture from other sources than the low moisture epoxidized natural fat or oil.

33. A process according to claim 32, wherein the first transesterification step is carried out until substantially completed.

34. A process according to claim 32, wherein both transesterification steps are carried out in the substantial absence of moisture from other sources than the low moisture epoxidized natural fat or oil.

35. A process according to claim 32, wherein at least the first transesterification step is additionally carried out under reduced pressure and with an inert gas atmosphere.

36. A process according to claim 35, wherein both transesterification steps are carried out under reduced pressure and with an inert gas atmosphere.
